(19) United States
(12) Patent Application Publication
(10) Pub. No.: US 2025/0352758 A1
(43) Pub. Date: Nov. 20, 2025

(54) METHOD FOR MANUFACTURING CATHETER

(71) Applicant: TERUMO KABUSHIKI KAISHA, Tokyo (JP)

(72) Inventors: Yuji MOTOSE, Hiratsuka-city (JP); Tsutomu SUGIKI, Fuji-city (JP)

(73) Assignee: TERUMO KABUSHIKI KAISHA, Tokyo (JP)

(21) Appl. No.: 19/279,742

(22) Filed: Jul. 24, 2025

Related U.S. Application Data
(63) Continuation of application No. PCT/JP2024/001585, filed on Jan. 22, 2024.

(30) Foreign Application Priority Data
Jan. 26, 2023 (JP) 2023-010123

(51) Int. Cl. A61M 25/00

(52) U.S. Cl. CPC A61M 25/0012; A61M 2205/0216

(57) ABSTRACT

A method for manufacturing a catheter, which includes, disposing a plurality of catheter members arranged in a predetermined manner via an insertion member inside a first hollow portion of an elastically deformable elastic body having the first hollow portion, the elastic body and the plurality of catheter members is moved inside a second hollow portion of a hard component to bring at least a part of an entire peripheral portion of an inner peripheral surface of the second hollow portion of the hard component into contact with (or adheres to) at least a part of an entire peripheral portion of an outer peripheral surface of the elastic body so as to apply an external force to the elastic body, irradiating the plurality of catheter members with a laser beam and fusing the plurality of catheter members arranged in a predetermined manner.

### CROSS-REFERENCES TO RELATED APPLICATIONS

This application is a continuation of International Application No. PCT/JP2024/001585 filed on Jan. 22, 2024, which claims priority to Japanese Application No. 2023-010123 filed on Jan. 26, 2023, the entire content of both of which is incorporated herein by reference.

### TECHNOLOGICAL FIELD

The present disclosure relates to a method for manufacturing a catheter.

### BACKGROUND DISCUSSION

When various therapeutic actions are performed in an organ of a living body, a medical instrument including a tubular body constituted by a flexible hollow tubular member is often used. Commonly known instruments of this type of medical instrument include a guiding catheter used for delivering a catheter device such as a balloon catheter to a desired position in a living body, a contrast catheter used for discharging a contrast medium into a living body, and a microcatheter used for discharging a medicine.

The above-described catheter may be manufactured through a joining of hollow tubes by a laser or the like. The above-described conventional technique of joining tubes to each other at a portion where the tubes are to be joined may employ a heat shrinkable tube (see Japanese Patent Application Publication No. 2002-301160 A).

The above-described heat shrinkable tube is disposed so as to cover a part of the catheter member in a state where the catheter member to be processed is disposed in a predetermined manner, and it is necessary to hold the catheter member in a contact state by applying heat to be thermally shrank, and to be removed from the catheter member after laser processing. However, since the removal operation of the heat shrinkable tube is manually performed by the operator, there is a risk that the catheter member is damaged, and it is necessary to redo the operation when the removal cannot be performed well. In addition, since the heat shrinkable tube shrinks after heating, the heat shrinkable tube cannot be reused and must be discarded after a single use.

As described above, in the method for manufacturing the catheter using the heat shrinkable tube, the influence on the manufacturing cost such as the labor cost required for the removal operation of the heat shrinkable tube and the material cost of the heat shrinkable tube is relatively large, and there is room for improvement.

In addition, International Patent Application Publication No. WO 2013/122083 A discloses a method for welding a catheter member that heats a welded portion while pressing the welded portion (for example, abutting surfaces of a plurality of catheter members) of the catheter member with a pressurizing tube without using a heat shrinkable tube.

However, when the catheter member is welded (connected) by the method disclosed in International Patent Application Publication No. WO 2013/122083 A, the following problems arise.

In the method disclosed in International Patent Application Publication No. WO 2013/122083 A, as a specific means for realizing compression and maintenance of the welded portion of the catheter member, a mechanism for inflating and compressing a part of the pressurizing tube like a balloon by sending air into a hollow housing in which the pressurizing tube is disposed and increasing the internal pressure in the hollow housing, and a mechanism for compressing and deforming the pressurizing tube by applying mechanical pressure to the pressurizing tube from both end sides in the axial direction are adopted.

In the mechanism as described above, it is not easy to uniformly pressurize the catheter member, and for example, there is a possibility of manufacturing a catheter in which the fusion surface of the catheter member is uneven. The manufacture of catheters with non-uniform fusions are often of poor quality.

### SUMMARY

A method is disclosed for manufacturing a catheter capable of saving labor and reducing cost while improving quality at the time of manufacturing the catheter by smoothly leveling a fusion surface of the catheter.

(1) A method for manufacturing a catheter including: in an external force application state in which, in a state where a plurality of catheter members arranged in a predetermined manner via an insertion member are disposed inside a first hollow portion of an elastically deformable elastic body having a laser transmission property and having the first hollow portion through which a plurality of catheter members can be inserted at no load, the elastic body and the plurality of catheter members arranged in the predetermined manner is moved inside a second hollow portion of a hard component having a laser transmission property and harder than the elastic body to bring at least a part of an entire peripheral portion of an inner peripheral surface of the second hollow portion of the hard component into contact with at least a part of an entire peripheral portion of an outer peripheral surface of the elastic body so as to apply an external force to the elastic body, irradiating the plurality of catheter members arranged in the predetermined manner via the hard component and the elastic body with a laser beam and fusing the plurality of catheter members arranged in the predetermined manner.

(2) The method for manufacturing a catheter according to (1), in which the second hollow portion of the hard component includes an inlet portion and an outlet portion positioned in a moving direction of the elastic body and the plurality of catheter members arranged in the predetermined manner, and the inlet portion has a larger cross-sectional area than the outlet portion.

(3) The method for manufacturing a catheter according to (2), in which a cross-sectional area of the inlet portion gradually decreases along the moving direction.

(4) The method for manufacturing a catheter according to (2) or (3), in which the laser beam is emitted toward a vicinity of the inlet portion of the hard component.

(5) The method for manufacturing a catheter according to any one of (1) to (4), in which a surface layer for reducing frictional resistance is provided on the outer peripheral surface of the first hollow portion of the elastic body and/or the inner peripheral surface of the hard component.

(6) The method for manufacturing a catheter according to any one of (1) to (5), in which the plurality of catheter members arranged in the predetermined manner include a first member and a second member arranged in a radial direction.

(7) The method for manufacturing a catheter according to any one of (1) to (5), in which the plurality of catheter members arranged in the predetermined manner include a first member and a second member arranged in an axial direction.

(8) A method for manufacturing a catheter including: in an external force conversion state in which, in a state where a plurality of catheter members arranged in a predetermined manner on an insertion member is disposed inside a first hollow portion of an elastically deformable elastic body having a laser transmission property and having the first hollow portion through which the plurality of catheter members can be inserted at no load, and the elastic body and the plurality of catheter members arranged in the predetermined manner are disposed inside a second hollow portion of a hard component which has a laser transmission property, has the second hollow portion located coaxially with the insertion member, and is harder than the elastic body, movement of the elastic body in a coaxial direction inside the second hollow portion of the hard component is converted into a radial external force which brings at least a part of an entire peripheral portion of an inner peripheral surface of the second hollow portion of the hard component into contact with at least a part of an entire peripheral portion of an outer peripheral surface of the elastic body and is applied to the plurality of catheter members arranged in the predetermined manner, irradiating predetermined portions of the plurality of catheter members arranged in a predetermined manner through the hard component and the elastic body with a laser beam, and fusing the predetermined portions.

(9) A method for manufacturing a catheter comprising: disposing via an insertion member, a plurality of catheter members arranged in a predetermined manner inside a first hollow portion of an elastically deformable elastic body having a laser transmission property; moving the elastic body and the plurality of catheter members arranged in the predetermined manner inside a second hollow portion of a hard component having a laser transmission property and hardness greater than the elastic body, and bringing at least a part of a peripheral portion of an inner peripheral surface of the second hollow portion of the hard component into contact with at least a part of a peripheral portion of an outer peripheral surface of the elastic body and to apply an external force to the elastic body; and irradiating the plurality of catheter members arranged in the predetermined manner via the hard component and the elastic body with a laser beam and fusing the plurality of catheter members arranged in the predetermined manner

According to the present disclosure, it is possible to reduce labor and cost while improving the quality of a catheter manufactured by fusing a plurality of catheter members.

### BRIEF DESCRIPTION OF THE DRAWINGS

FIG. 1 is a schematic configuration diagram of a catheter manufactured by a method for manufacturing a catheter according to the present disclosure.

FIG. 2 is a view schematically illustrating a manufacturing device used in the method for manufacturing the catheter according to the embodiment.

FIG. 3 is a cross-sectional view of a hard component included in the manufacturing device according to the embodiment.

FIG. 4 is a flowchart illustrating the method for manufacturing the catheter according to the embodiment.

FIG. 5A is a view for explaining the method for manufacturing the catheter according to the embodiment.

FIG. 5B is a view for explaining the method for manufacturing the catheter according to the embodiment.

FIG. 5C is a view for explaining the method for manufacturing the catheter according to the embodiment.

FIG. 5D is a view for explaining the method for manufacturing the catheter according to the embodiment.

FIG. 5E is a view for explaining the method for manufacturing the catheter according to the embodiment, and a view illustrating cross-sectional views of an A-A cross section and a B-B cross section of the manufacturing device.

FIG. 5F is a view for explaining the method for manufacturing the catheter according to the embodiment.

FIG. 6 is an enlarged view of a broken line portion 6A illustrated in FIG. 5F.

FIG. 7A is a view illustrating a method for manufacturing the catheter according to a first configuration example.

FIG. 7B is a view illustrating a part of the catheter according to the first configuration example.

FIG. 8A is a view illustrating a method for manufacturing a catheter according to a second configuration example.

FIG. 8B is a view illustrating a part of the catheter according to the second configuration example.

FIG. 9A is a view illustrating a method for manufacturing a catheter according to a third configuration example.

FIG. 9B is an axis-orthogonal cross-sectional view illustrating a part of the catheter according to the third configuration example.

FIG. 9C is an axis-orthogonal cross-sectional view illustrating a part of the catheter according to the third configuration example.

### DETAILED DESCRIPTION

Hereinafter, a mode for carrying out the present disclosure will be described in detail with reference to the drawings. Embodiments herein described are illustrated to embody the technical idea of the present disclosure and do not limit the present disclosure. Other feasible embodiments, examples, operation technologies and the like that could be conceived by those skilled in the art without departing from the gist of the present disclosure are all included in the scope and gist of the present disclosure and included in the disclosure recited in claims and the scope of equivalents thereof.

For convenience of illustration and ease of understanding, the drawings attached to the present specification may be schematically represented by changing a scale, an aspect ratio, a shape, and the like, from actual ones as appropriate, but are merely examples, and do not limit the interpretation of the present disclosure.

In the present specification, the following directions are defined for convenience of description. The “axial direction” of a catheter body 10 and a catheter member 30 means a longitudinal direction in which the catheter body 10 and the catheter member 30 extend, and is a direction along the “central axis C” illustrated in the drawing. In the present specification, a direction along the same direction as the above-described “axial direction” is also referred to as a “coaxial direction”.

In addition, the “radial direction” of the catheter body 10 and the catheter member 30 is a direction vertically away from or approaching the central axis C. The “circumferential direction” of the catheter body 10 and the catheter member 30 is a direction of a set of points separated by a predetermined distance with respect to the central axis C.

Note that, in the following description, ordinal numerals such as “first” and “second” will be given, but are used for convenience and do not define any order unless otherwise specified.

A catheter 100 manufactured by the method for manufacturing a catheter according to the present embodiment may be inserted into a blood vessel, a bile duct, a trachea, an esophagus, a urethra, or other cavities or lumens in a living body to be used for treatment, diagnosis, or the like. The catheter 100 can be, for example, a balloon catheter, a microcatheter, a contrast catheter, a guiding catheter, or the like for percutaneous transluminal coronary angioplasty (PTCA) or percutaneous transluminal angioplasty (PTA). In addition, the present disclosure can be appropriately used for a device described in a configuration example described later.

### Catheter

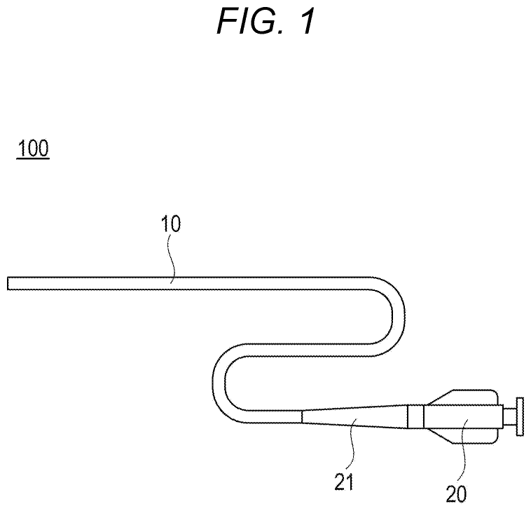

FIG. 1 is a schematic diagram illustrating the catheter 100 manufactured by the method for manufacturing a catheter according to an embodiment of the present disclosure.

As illustrated in FIG. 1, the catheter 100 is configured to provide an elongated catheter body 10 that can be introduced into a living body, a hub 20 connected to a proximal end portion of the catheter body 10, and an anti-kink protector 21 in the vicinity of a connecting portion between the catheter body 10 and the hub 20. Note that the specific configuration of the catheter 100 is not particularly limited, and for example, the anti-kink protector 21 may not be provided.

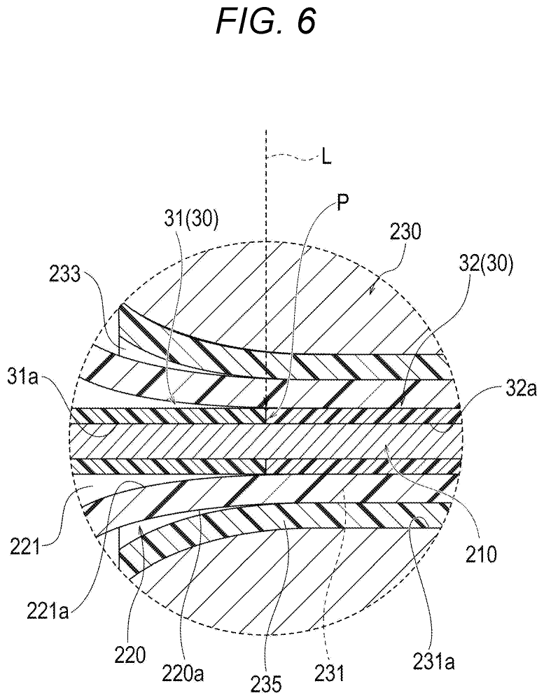

The catheter body 10 is configured as a flexible tubular member in which a lumen extending in the axial direction is formed. As will be described later in the present embodiment, the catheter body 10 is formed by fusing an adjacent portion P where the end portion of a first member 31 and the end portion of a second member 32 are abutted and brought into contact with each other in a state where two cylindrical members of the first member 31 and the second member 32 are arranged in the axial direction as the catheter member 30 (see FIG. 6). The adjacent portion P is provided at one end portion of the first member 31 and the second member 32 in the axial direction.

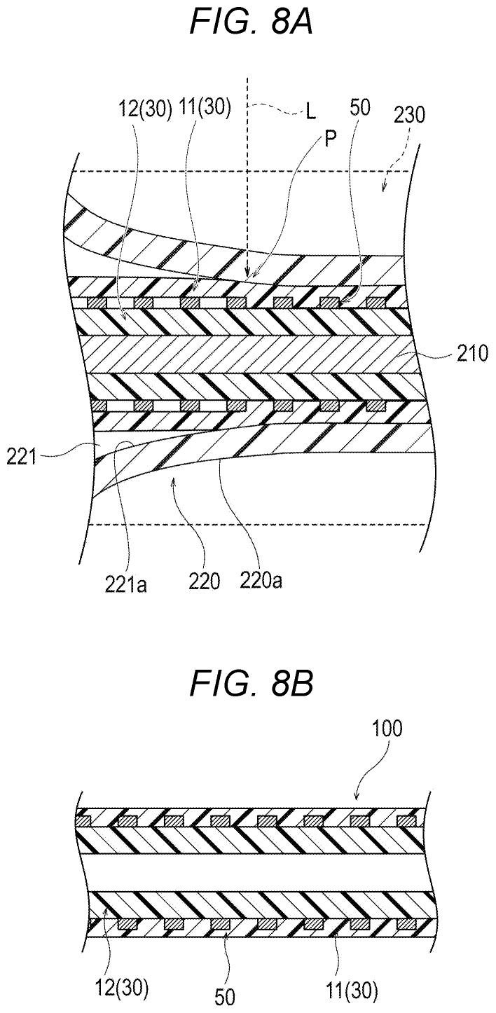

Examples of the constituent material of the first member 31 and the second member 32 can include, in addition to a polyamide resin, a polyester resin, a polyolefin resin, and a polyurethane resin, a polyamide elastomer, a polyester elastomer, a polyurethane elastomer, or a mixture of one or more of the above-mentioned constituent materials of the first member 31 and the second member 32 or a mixture of two or more of the above-mentioned constituent materials of the first member 31 and the second member 32 having different hardness. As a component of the catheter member 30, three or more members configured by various resins or elastomers having different hardness may be arranged so as to be flexible from the proximal end toward the distal end. In addition, as a component of the catheter member 30, in order to harden a predetermined portion and soften the other portion, members configured by resin or elastomer having hardness corresponding to the portion may be arranged. These members of resin or elastomer and arranged by corresponding hardness can be used as, for example, a first tube to be an outer layer in a second configuration example (see FIGS. 8A and 8B) to be described later after fusion bonding. Further, in at least one of the first member 31 and the second member 32, a resin layer having higher slidability than each resin material exemplified above, for example, a layer configured by polytetrafluoroethylene resin may be provided inside in order to enhance the slidability of the inner surface.

The constituent materials of the first member 31 and the second member 32 preferably include the same kind of constituent materials. As an example, a constituent material of the first member 31 is a polyamide elastomer, and a constituent material of the second member 32 is a polyamide resin. The first member 31 and the second member 32 may be configured by materials different from the above materials. As an example, the constituent material of the first member 31 may be a polyester elastomer, and the constituent material of the second member 32 may be a polyester resin. As long as a predetermined bonding strength or more can be obtained as described above, the first member 31 and the second member 32 may be configured by different materials.

In addition, the first member 31 and the second member 32 can contain a pigment or dye that develops white, black, blue, red, yellow, or the like, and a mixture of the pigment or dye that develops white, black, blue, red, and/or yellow. Such a pigment or dye may be selected from materials that absorb a laser beam L and generate heat. Examples of the material of the pigment or dye that generates heat by absorbing a laser beam L can include, for example, carbon black.

The first member 31 and the second member 32 can be configured to include a powdered X-ray contrast material. Specific examples of the powdered X-ray contrast material can include, for example, compounds of gold, titanium, bismuth, and tungsten. Furthermore, in the first member 31 and the second member 32, a reinforcing body configured by tungsten, SUS (i.e., stainless steel), or the like may be disposed in the above-described constituent material. As the reinforcing body, for example, a coiled or blade-shaped reinforcing body can be used.

The hub 20 is liquid-tightly fixed to the catheter body 10 with an adhesive, a fixing tool, or the like. The hub 20 can be used as a port through which a guide wire is inserted into the lumen of the catheter body 10, or an injection port through which a liquid medicine, an embolic substance, a contrast medium, or the like is injected into the lumen, and also can be used as a grip portion when the catheter 100 is operated. Examples of a material usable for the hub 20 can include a thermoplastic resin such as polycarbonate, polyamide, polysulfone, or polyarylate.

Note that, when the catheter 100 has the anti-kink protector 21 as illustrated in FIG. 1, the anti-kink protector 21 can be configured by an elastic material provided so as to surround a part of the proximal end portion of the catheter body 10. As a constituent material of the anti-kink protector 21, natural rubber, silicone resin, or the like can be used, for example.

### Manufacturing Device

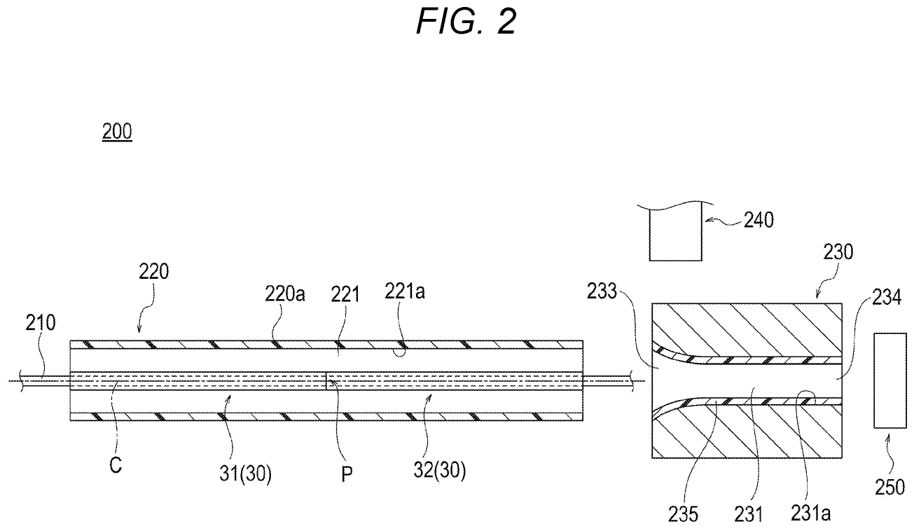

Next, a catheter manufacturing device 200 (hereinafter, also simply referred to as “manufacturing device 200”) according to an embodiment will be described. FIG. 2 is a schematic configuration diagram of the manufacturing device 200.

The manufacturing device 200 includes an insertion member 210, an elastic body 220, a hard component 230, a laser irradiation unit 240, and a holding mechanism 250.

### Insertion Member

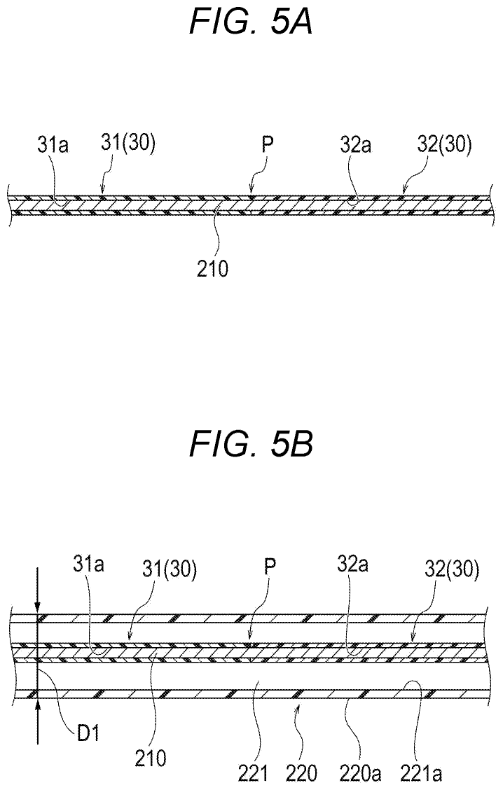

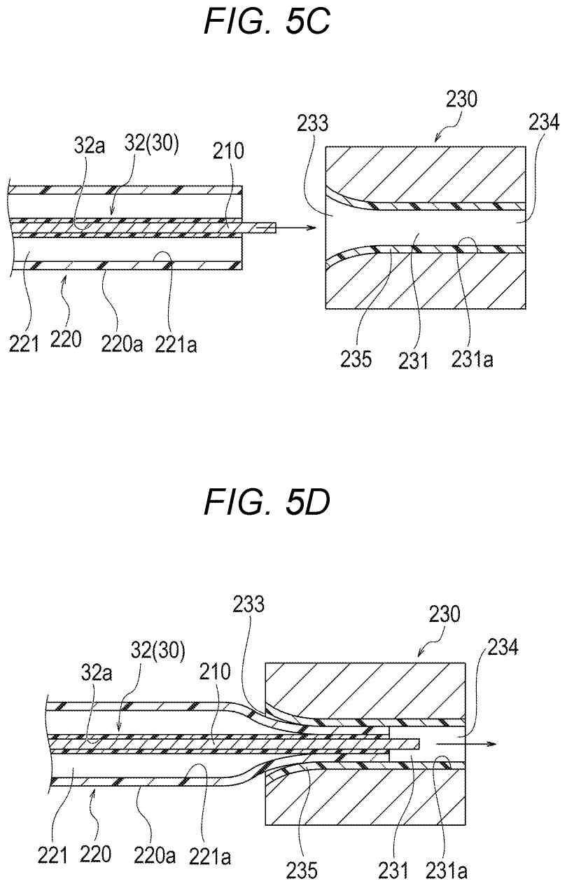

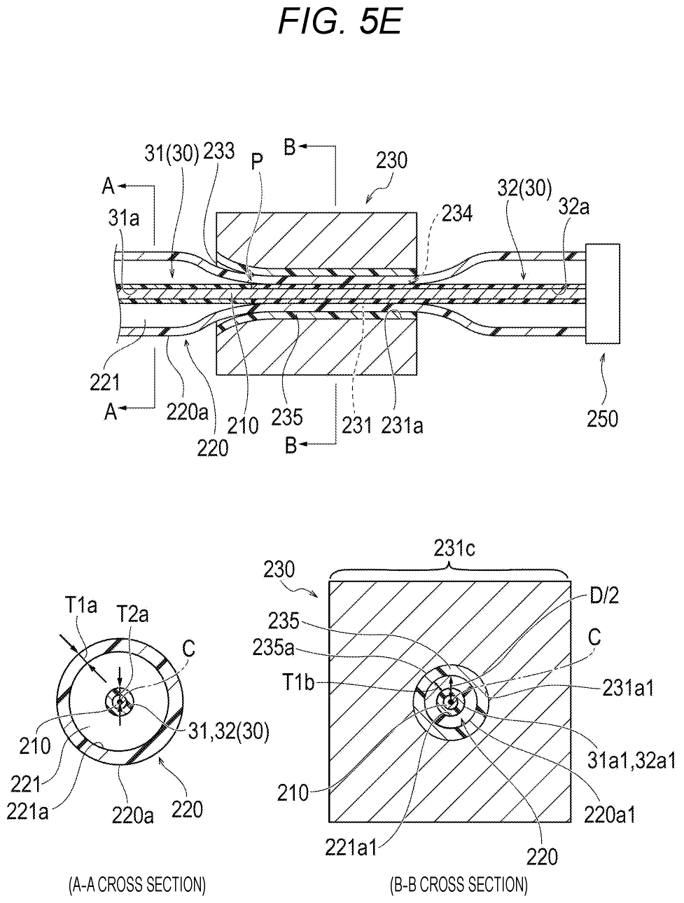

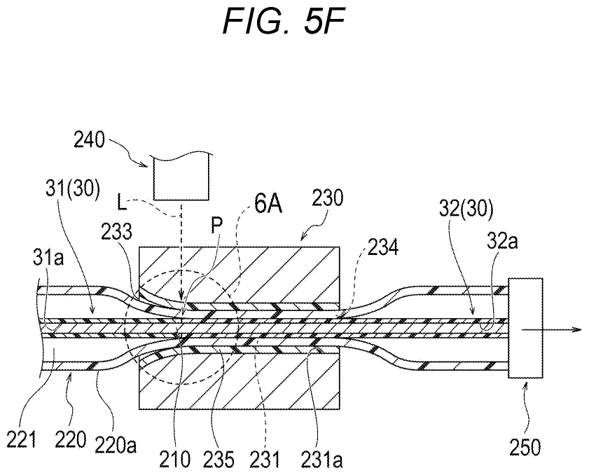

The insertion member 210 is configured to be insertable through a lumen 31a of the cylindrical first member 31 and a lumen 32a of the second member 32 to be fused (see FIG. 5A).

The insertion member 210 can be configured by, for example, a core metal. The insertion member 210 can be configured by a metal material or the like having heat resistance when the first member 31 and the second member 32 are fused and rigidity capable of holding the positions of the members 31 and 32.

The insertion member 210 can be configured by a cylindrical member having a circular cross-sectional shape intersecting the axial direction. However, the cross-sectional shape, the material, the length, the outer diameter, and the like of the insertion member 210 are not particularly limited as long as the insertion member has a configuration in which the first member 31 and the second member 32 can be inserted. The insertion member 210 may be configured by, for example, a hollow tubular member.

### Elastic Body

As illustrated in FIG. 5B, the elastic body 220 includes a first hollow portion 221 through which the first member 31 and the second member 32 attached to the insertion member 210 can be inserted.

As illustrated in FIGS. 5F and 6, the elastic body 220 is configured to be elastically deformable so as to be able to apply a force inward in the radial direction to the adjacent portion P between the first member 31 and the second member 32. Specifically, the elastic body 220 is configured to be elastically deformable so as to be able to apply a force to the adjacent portion P in a direction toward the central axis C. The elastic body 220 maintains a predetermined cylindrical shape as illustrated in FIG. 5B at no load, and deforms to reduce the diameter when an external force is applied via the hard component 230. The elastic body 220 is configured to form a gap between the first member 31 and the second member 32 disposed in the first hollow portion 221 at no load.

As illustrated in FIGS. 5E, 5F, and 6, when an external force is applied from the hard component 230, the elastic body 220 is deformed inward in the radial direction and comes into contact with the outer peripheral surface of the first member 31 and the outer peripheral surface of the second member 32. In this state, the elastic body 220 applies a pressing force to the adjacent portion P between the first member 31 and the second member 32, and holds a state in which both the members 31 and 32 abut on each other at the adjacent portion P. In the present specification, a state in which the hard component 230 applies an external force to the catheter member 30 via the elastic body 220 so that the elastic body 220 is displaced toward a portion (adjacent portion P) to be fused is referred to as an “external force application state”.

In addition, in the method for manufacturing a catheter according to the present embodiment, as illustrated in FIG. 5E, the elastic body 220 and the catheter members 30 are disposed in the second hollow portion 231 of the hard component 230 in a state where the catheter members 30 arranged in a predetermined manner through the insertion member 210 are disposed inside the first hollow portion 221 of the elastic body 220. Then, the movement of the elastic body 220 in the coaxial direction inside the second hollow portion 231 of the hard component 230 is converted into a radial external force applied to the plurality of catheter members 30 arranged in a predetermined manner in which at least a part of an inner peripheral surface 231a of the second hollow portion 231 of the hard component 230 is brought into contact with (or adheres to) at least a part of an outer peripheral surface 220a of the elastic body 220, and the “external force conversion state” is established.

In the external force conversion state, the hard component 230 applies a force to the elastic body 220 in a direction of reducing the diameter of the first hollow portion 221 of the elastic body 220. Therefore, by bringing the above-described external force conversion state into a state in which the adjacent portions P of the plurality of catheter members 30 are disposed in the first hollow portion 221 of the elastic body 220, it is possible to create a state substantially the same as the external force application state in an arbitrary range in the axial direction of the catheter member 30. Therefore, by irradiating the adjacent portion P with the laser beam L in the external force conversion state, the members 31 and 32 can be appropriately fused to each other in a state where an external force is applied to the adjacent portion P. As described above, the elastic body 220 can also be referred to as an external force conversion member that converts the coaxial movement of the elastic body 220 inside the second hollow portion 231 of the hard component 230 into a radial external force applied to the plurality of catheter members 30 arranged in a predetermined manner.

The elastic body 220 can be configured to include a material having a laser transmission property that enables the adjacent portion P to be fused when the laser beam L is emitted from the outside of the hard component 230 toward the adjacent portion P disposed inside the first hollow portion 221 and the second hollow portion 231. In the present specification, the “laser transmission property” means that the elastic body 220 contains a material having a transmittance of 80% or more with respect to the laser beam L per 1 mm of the thickness of the elastic body in the radial direction.

The elastic body 220 can contain a material having higher heat resistance than the first member 31 and the second member 32. As a material of the elastic body 220, for example, silicone rubber, fluororubber, or the like can be used.

### Hard Component

The hard component 230 has a laser transmission property. In addition, the hard component 230 can be configured by a member harder than the elastic body 220.

Similar to the elastic body 220, the laser transmission property of the hard component 230 means that the hard component 230 is configured to have transmittance of 80% per 1 mm of the thickness of the elastic body in the radial direction. As a constituent material of the hard component 230, for example, a material having a laser transmission property and harder than the elastic body 220 is suitable. Furthermore, the constituent material of the hard component 230 is more preferably a material that hardly deforms due to heat transfer from the workpiece that generates heat by irradiation with the laser beam L. As such a material for the hard component 230, quartz glass or the like can be suitably used.

As illustrated in FIG. 5F, the hard component 230 has a second hollow portion 231 in which the elastic body 220 and the members 31 and 32 are disposed when the members 31 and 32 are fused. When the adjacent portion P of the elastic body 220 is fused, at least a part of the elastic body 220 in the axial direction is disposed inside the second hollow portion 231.

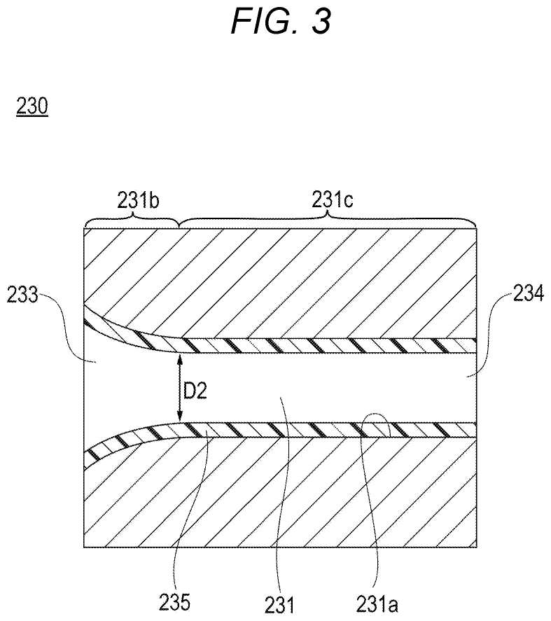

As illustrated in FIGS. 3, 5D, and 5E, the second hollow portion 231 of the hard component 230 has an inlet portion 233 and an outlet portion 234 positioned in the moving direction (hereinafter, also simply referred to as “moving direction”) of the members 31 and 32 arranged in the elastic body 220 and the insertion member 210.

The elastic body 220 and the members 31 and 32 are inserted into the second hollow portion 231 via the inlet portion 233. In addition, a part or the whole of the elastic body 220 and the members 31 and 32 is taken out to the outside of the second hollow portion 231 via the outlet portion 234 located on the opposite side of the inlet portion 233. In the present embodiment, as illustrated in FIGS. 5F and 6, when the members 31 and 32 are fused, a predetermined range in the axial direction including the adjacent portions P of the members 31 and 32 is disposed inside the second hollow portion 231.

As illustrated in FIGS. 2, 3, and 5D, the second hollow portion 231 of the hard component 230 is configured such that the cross-sectional area gradually decreases along the moving direction of the members 31 and 32 arranged in the elastic body 220 and the insertion member 210. That is, the inlet portion 233 includes a cross-sectional area decreasing portion 231b having a cross-sectional shape in which the cross-sectional area gradually decreases from the opening position (left end in FIG. 3) of the inlet portion 233 toward the outlet portion 234 side (right side in FIG. 3). For example, the second hollow portion 231 can be configured to have a cross-sectional shape in which the cross-sectional area gradually decreases between 0.5 mm and 250 mm within a range of 1 mm to 500 mm from the opening position of the inlet portion 233 toward the outlet portion 234 side. In addition, the hard component 230 includes a constant cross-sectional area portion 231c provided between the cross-sectional area decreasing portion 231b and the outlet portion 234 side.

In the example illustrated in FIG. 3, a curved cross-sectional shape in which the cross-sectional area of the inlet portion 233 gradually decreases toward the outlet portion 234 side is illustrated, but the cross-sectional shape of the inlet portion 233 is not limited to such a shape. For example, the inlet portion 233 may have a linear cross-sectional shape in which the cross-sectional area of the inlet portion 233 gradually decreases toward the outlet portion 234 side. Further, for example, the inlet portion 233 may be formed such that the cross-sectional area of the inlet portion 233 is substantially constant along the moving direction. As in the present embodiment, by having the cross-sectional shape in which the cross-sectional area gradually decreases from the opening position of the inlet portion 233 toward the outlet portion 234 side, the elastic body 220 can smoothly transition to the second hollow portion 231 of the hard component 230, and it is possible to suppress generation of uneven force on the outer surfaces of the members 31 and 32.

In addition, the cross-sectional shape of the second hollow portion 231 of the hard component 230 is not particularly limited as long as an external force can be applied to the elastic body 220 and the members 31 and 32 disposed inside the second hollow portion 231. For example, the cross-sectional shape of the second hollow portion 231 of the hard component 230 can be any shape according to the cross-sectional shape of the elastic body 220 and the cross-sectional shapes of the members 31 and 32. For example, the second hollow portion 231 of the hard component 230 may be formed such that portions having a small cross-sectional area are intermittently formed at different portions in the moving direction, or such that the cross-sectional area near the outlet portion 234 gradually increases toward the moving direction side.

In the second hollow portion 231, an inner diameter D2 (see FIG. 3) of a portion around a portion where the adjacent portion P is disposed when the members 31 and 32 are fused is smaller than an outer diameter D1 (see FIG. 5B) of the elastic body 220 in the unloaded state. Therefore, as illustrated in FIG. 5E, when the adjacent portion P is disposed at a predetermined position of the second hollow portion 231, the inner peripheral surface 231a of the second hollow portion 231 of the hard component 230 abuts on the outer peripheral surface 220a of the elastic body 220, and the diameter of the elastic body 220 is reduced. As a result, the elastic body 220 enters an external force application state. The elastic body 220 maintains the external force application state while the elastic body 220 is disposed in the second hollow portion 231. The inner diameter D2 is the inner diameter of a surface layer 235 when the surface layer 235 is provided on the inner peripheral surface 231a of the second hollow portion 231. In a case where the surface layer 235 is provided on the inner peripheral surface 231a of the second hollow portion 231, the “inner peripheral surface of the second hollow portion” is read as the inner peripheral surface of the surface layer 235.

More specifically, as illustrated in the A-A cross-sectional view and the B-B cross-sectional view of FIG. 5E, a thickness T1a of the elastic body 220 at no load is larger than the thickness T1b of the elastic body 220 when disposed in the constant cross-sectional area portion 231c of the second hollow portion 231 of the hard component 230. That is, a radius D/2 (in the present embodiment, it corresponds to the radius of the inner peripheral surface of the surface layer 235) of the constant cross-sectional area portion 231c of the second hollow portion 231 is smaller than the total thickness obtained by adding a radius T2a of the catheter member 30 before being disposed in the second hollow portion 231 of the hard component 230 and the thickness T1a of the elastic body 220 at no load. With this relationship, the inner peripheral surface 231a of the second hollow portion 231 of the hard component 230 is brought into contact with (or adheres to) the outer peripheral surface 220a of the elastic body 220 to apply an appropriate pressure to the catheter member 30.

The second hollow portion 231 of the hard component 230 restricts deformation of the elastic body 220 outward in the radial direction in a state where the elastic body 220 is disposed in the second hollow portion 231. As a result, the relative movement between the hard component 230 and the elastic body 220 is converted into deformation toward the radial inside of the elastic body 220. Since this radially inner deformation is converted into a predetermined pressure applied to the members 31 and 32, a smooth fusion surface can be formed in the members 31 and 32 by applying thermal energy in this state as described later.

The terminal end (that is, the position where the decrease in the cross-sectional area of the inlet portion 233 ends) of the inlet portion 233 of the second hollow portion 231 has substantially the same diameter as the predetermined position where the adjacent portion P of the second hollow portion 231 is disposed when the members 31 and 32 are fused. Therefore, as illustrated in FIG. 5E, when the elastic body 220 and the members 31 and 32 are inserted into the second hollow portion 231 via the inlet portion 233 and the elastic body 220 and the members 31 and 32 are moved toward the outlet portion 234, the elastic body 220 receives a restraining force from the inner peripheral surface 231a of the second hollow portion 231 to the outer peripheral surface 220a, and is deformed so as to gradually reduce the diameter from the distal end side in the moving direction (distal end side in the axial direction). By inserting the elastic body 220 and the members 31 and 32 into the second hollow portion 231 by a predetermined length in the axial direction via the inlet portion 233 and further moving the elastic body 220 and the members 31 and 32 by a predetermined length along the axial direction, a certain range of the adjacent portion P and the periphery thereof can be brought into the external force application state. In addition, in a state where the elastic body 220 and the members 31 and 32 are disposed inside the second hollow portion 231, by maintaining the axial positions of the elastic body 220 and the members 31 and 32, a certain range of the adjacent portion P and the periphery thereof can be appropriately and reliably maintained in the external force application state. As described above, the hard component 230 can also be referred to as an external force applying member that restricts the deformation of the elastic body 220 outward in the radial direction and applies an external force to the elastic body 220.

As illustrated in FIG. 3, the hard component 230 can be configured to have the surface layer 235 for reducing frictional resistance (sliding resistance with respect to the elastic body 220) on the inner peripheral surface 231a of the second hollow portion 231.

The constituent material of the surface layer 235 is not particularly limited, but preferably has heat resistance in addition to reducing the frictional resistance as described above. Such a material of the surface layer 235 can be configured by, for example, a fluororesin such as a polytetrafluoroethylene resin. From the viewpoint of friction resistance, the constituent material of the surface layer 235 can be configured by a polyurethane elastomer resin. The surface layer 235 may be disposed only on the outer peripheral surface 220a of the elastic body 220, or may be disposed on both the outer peripheral surface 220a of the elastic body 220 and the inner peripheral surface 231a of the second hollow portion 231. In addition, the surface layer 235 is preferably configured by a material having a laser transmission property.

Regarding the matter that an entire peripheral portion 231a1 of at least a part of the inner peripheral surface 231a of the second hollow portion 231 of the hard component 230 is brought into contact with (or adheres to) an entire peripheral portion 220a1 of at least a part of the outer peripheral surface 220a of the elastic body 220 to apply an external force to the elastic body 220, in the embodiment exemplified in FIGS. 5A to 5F, an aspect in which all the entire peripheral portion 231a1 of the inner peripheral surface 231a of the second hollow portion 231 of the hard component 230 in the axial direction is brought into contact with (or adheres to) a part of the entire peripheral portion 220a1 of the outer peripheral surface 220a of the elastic body 220 in the axial direction to apply an external force to the elastic body 220 has been described. However, the present disclosure is not limited to such an aspect, and an aspect in which a part of the entire peripheral portion 231a1 of the inner peripheral surface 231a of the second hollow portion 231 of the hard component 230 in the axial direction is brought into contact with (or adheres to) an entirety of the peripheral portion 220a1 of the outer peripheral surface 220a of the elastic body 220 in the axial direction to apply an external force to the elastic body 220 can also be set as follows. That is, this can be realized by making the axial length of the elastic body 220 shorter than the axial length of the second hollow portion 231 of the hard component 230. Furthermore, for example, it is preferable that an outer diameter of the holding mechanism 250 described later is set to a size that allows insertion into the second hollow portion 231 of the hard component 230. This aspect is suitable, for example, in a case where the processed portion is short. In this aspect in which the processed portion, the processing time can be shortened and the handling of the elastic body 220 can be simplified.

An aspect in which all the entire peripheral portion 231a1 of the inner peripheral surface 231a of the second hollow portion 231 of the hard component 230 in the axial direction is brought into contact with (or adheres to) an entirety of the peripheral portion 220a1 of the outer peripheral surface 220a of the elastic body 220 in the axial direction to apply an external force to the elastic body 220 can also be realized by making the axial length of the elastic body 220 the same as the axial length of the second hollow portion 231 of the hard component 230. Even in a case where such a configuration is adopted, the same effects as those described above can be obtained.

An aspect in which a part of the entire peripheral portion 231a1 of the inner peripheral surface 231a of the second hollow portion 231 of the hard component 230 in the axial direction is brought into contact with (or adheres to) a part of the entire peripheral portion 220a1 of the outer peripheral surface 220a of the elastic body 220 in the axial direction to apply an external force to the elastic body 220 can also be set as follows. That is, the cross-sectional area and the inner diameter of the inlet portion 233 of the second hollow portion 231 of the hard component 230 can be set to be larger than the cross-sectional area and the outer diameter of the outer peripheral surface 220a of the elastic body 220 at no load, or the same structure can be provided in the outlet portion 234 of the second hollow portion 231 of the hard component 230. By providing such a configuration in the inlet portion 233 and the outlet portion 234, the diameter reduction and the diameter expansion of the elastic body 220 become smooth, and it is possible to suppress a trouble that may occur when the elastic body 220 moves in the second hollow portion 231 of the hard component 230 in advance.

### Laser Irradiation Unit

The laser irradiation unit 240 is used when the adjacent portion P and the like are fused via the elastic body 220 and the hard component 230. The laser irradiation unit 240 includes a light source that oscillates a laser beam L, a galvano scan (scanner) that changes the laser beam L oscillated from the light source in a predetermined direction by a motor, a mirror, or the like, a prism, and the like. One or a plurality of the laser irradiation units 240 are disposed at positions (around the hard component 230) where the adjacent portion P to be the fusion target portion of the catheter member 30 can be irradiated with the laser beam L.

The laser irradiation unit 240 emits the laser beam L having a wavelength that causes a fusion target portion such as the adjacent portion P to generate heat by radiation heating. The spot diameter of the laser beam L can be set to φ0.1 mm to φ10 mm, and the wavelength of the laser beam L can be set to 800 nm to 10,000 nm. When the laser beam L is emitted toward the adjacent portion P, the laser beam L passes through the hard component 230 and the elastic body 220, and forms a fusion bonded portion in the adjacent portion P.

As the fusion target portion irradiated with the laser beam L, a portion to be processed in the catheter member 30 can be arbitrarily selected. For example, in a state where a plurality of members to be the catheter member 30 are disposed side by side in the axial direction or the radial direction, end surfaces of the members and a peripheral portion of the end surfaces, a portion overlapping each other in a state where the two members are partially overlapped, a peripheral portion of the portion overlapping each other in state where the two members are partially overlapped, and the like can be set as the fusion target region.

### Holding Mechanism

As illustrated in FIG. 2, the manufacturing device 200 includes a holding mechanism 250 for controlling axial movement and/or circumferential movement (rotation) of the first member 31 and the second member 32 when processing the catheter member 30.

The holding mechanism 250 can be configured by, for example, a chuck (bearing jig). The manufacturing device 200 can be configured to control operation of the holding mechanism 250 via a motor and a gear. By causing the holding mechanism 250 to hold the end portion of the elastic body 220, the end portion of the catheter member 30, and the end portion of the insertion member 210, these members can be set in the manufacturing device 200 in a state of being movable in the axial direction and the circumferential direction. By operating the holding mechanism 250, it is possible to appropriately insert the elastic body 220 and the members 31 and 32 into the second hollow portion 231 of the hard component 230, move the adjacent portion P of the catheter member 30 after fusion, and remove (or take out) the members 31 and 32 from the second hollow portion 231 of the hard component 230.

In the example illustrated in FIG. 2, the manufacturing device 200 includes one holding mechanism 250 disposed on the outlet portion 234 side of the hard component 230. However, in the manufacturing device 200, the holding mechanism 250 may be disposed on the inlet portion 233 side of the hard component 230. In the manufacturing device 200, the holding mechanism 250 may be disposed on the inlet portion 233 side and the outlet portion 234 side of the hard component 230. In a case where the holding mechanism 250 is provided on the inlet portion 233 side and the outlet portion 234 side, it is preferable that the operation of the holding mechanism 250 is controlled so that the movements of the inlet portion 233 side and the outlet portion 234 side are synchronized.

### Method for Manufacturing Catheters

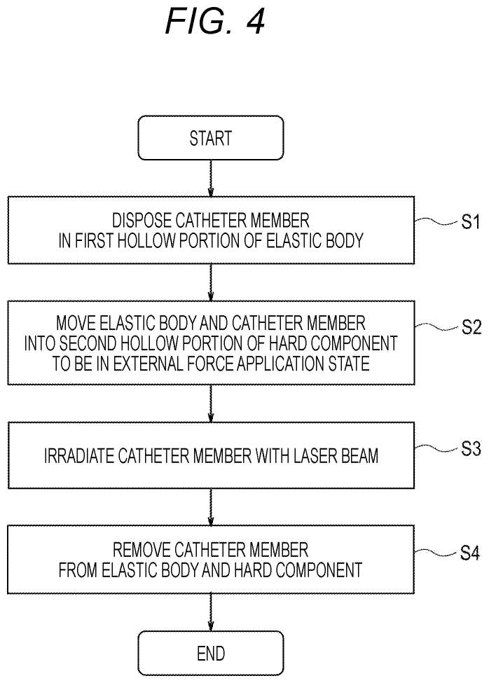

Next, a method for manufacturing a catheter according to the present embodiment will be described. FIG. 4 is a flowchart illustrating a method for manufacturing the catheter 100, and FIGS. 5A to 5F and FIG. 6 are views schematically illustrating each step of the method for manufacturing the catheter including a preparation stage.

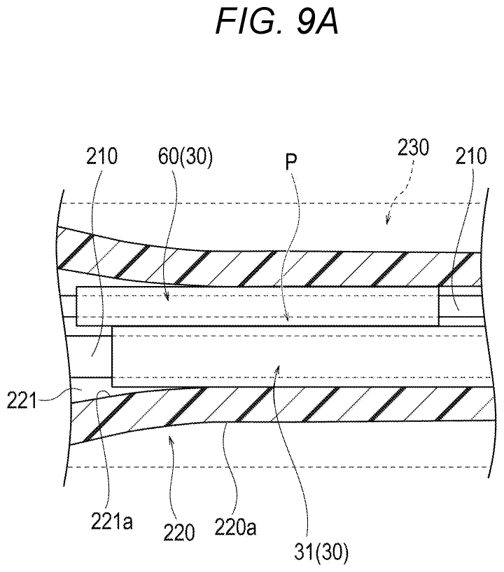

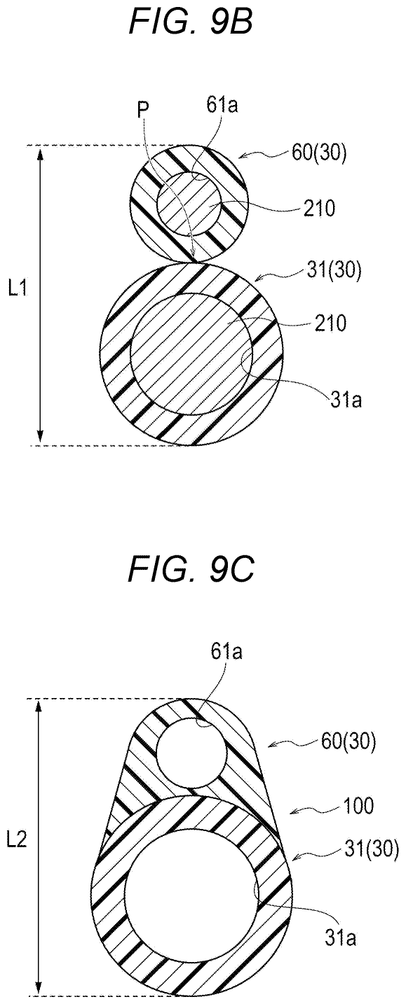

As illustrated in FIG. 5A, the plurality of catheter members 30 (the first member 31 and the second member 32) arranged in a predetermined manner via the insertion member 210 is prepared. Note that, in the present specification, “arranged in a predetermined manner via the insertion member 210” includes not only a state in which the members 31 and 32 are coaxially disposed via one insertion member 210 as shown in the present embodiment, but also a state in which a relative positional relationship between the members 31 and 32 inserted into one or a plurality of insertion members 210 is arranged at different positions in a radial direction or an axial direction with respect to a position of the insertion member 210 as shown in a configuration example and the like to be described later (see FIGS. 8A and 9A).

As illustrated in FIG. 5B, the plurality of catheter members 30 arranged in a predetermined manner via the insertion member 210 are disposed inside the first hollow portion 221 of the elastic body 220. In this state, the first member 31 and the second member 32 are not applied with an external force by the hard component 230, and are integrally movable and rotatable with the insertion member 210.

Next, as illustrated in FIGS. 5C and 5D, the elastic body 220 and the plurality of catheter members 30 arranged in a predetermined manner are moved into the second hollow portion 231 of the hard component 230.

Next, after the elastic body 220 and a part of the catheter member 30 are taken out from the outlet portion 234 side of the hard component 230, the end portions of the elastic body 220 and the catheter member 30 are held by the holding mechanism 250. When the catheter member 30 is held by the holding mechanism 250, it is possible to control the axial movement and/or rotation operation using the holding mechanism 250.

As illustrated in FIG. 5E, the plurality of catheter members 30 arranged in a predetermined manner via the insertion member 210 are disposed inside the first hollow portion 221 of the elastic body 220 (S1 in FIG. 4). As illustrated in FIG. 5F, the elastic body 220 and the plurality of catheter members 30 arranged in a predetermined manner move inside the second hollow portion 231 of the hard component 230 by the axial movement (see the arrow) using the holding mechanism 250 (S2 in FIG. 4). As a result, at least a part of the inner peripheral surface 231a of the second hollow portion 231 of the hard component 230 is brought into contact with (or adheres to) at least a part of the outer peripheral surface 220a of the elastic body 220, and an external force is applied to the elastic body 220.

In the manufacturing method of the present embodiment, since both the elastic body 220 and the plurality of catheter members 30 arranged in a predetermined manner move inside the second hollow portion 231 of the hard component 230, an external force is applied from the inner surface of the second hollow portion 231, but since frictional resistance when the catheter member 30 moves occurs on the outer peripheral surface 220a of the elastic body 220, there is no direct influence of the frictional resistance on the outer surface of the catheter member 30, or the frictional resistance is extremely small. As a result, the fusion surface (outer surface of abutting surface against which end portions of the members 31 and 32 abut each other) of the catheter member 30 is smoothly leveled.

As illustrated in the cross-sectional views taken along line A-A and line B-B in FIG. 5E, the insertion member 210 and the second hollow portion 231 of the hard component 230 are coaxially positioned. That is, the insertion member 210 and the second hollow portion 231 of the hard component 230 are disposed around the central axis C. Furthermore, the plurality of catheter members 30 and the first hollow portion 221 of the elastic body 220 at no load are coaxially positioned.

At least a part of the entire peripheral portion (at least a part of the entire peripheral portion in the moving direction) 231a1 of the inner peripheral surface 231a of the second hollow portion 231 of the hard component 230 is brought into contact with (or adheres to) at least a part of the entire peripheral portion (at least a part of the entire peripheral portion in the moving direction) 220a1 of the outer peripheral surface 220a of the elastic body 220 to be in a state where an external force is applied to the elastic body 220. Describing in detail in the B-B cross section of FIG. 5E, an outer surface 235a of the surface layer 235 of the constant cross-sectional area portion 231c of the second hollow portion 231 of the hard component 230 (the inner peripheral surface 231a of the second hollow portion 231) is brought into contact (or adheres to) with the entire peripheral portion 220a1 of the outer peripheral surface 220a of the elastic body 220 located in the constant cross-sectional area portion 231c of the second hollow portion 231 of the hard component 230. With such a configuration, the shape of the inner peripheral surface 231a of the second hollow portion 231 in the constant cross-sectional area portion 231c of the hard component 230 stabilizes the external force application state to the elastic body 220.

Furthermore, the stable external force application state contributes to smooth leveling of the fusion surface of the catheter member 30.

In the external force application state, an entire peripheral portion 221a1 of an inner peripheral surface 221a of the first hollow portion 221 of the elastic body 220 located in the constant cross-sectional area portion 231c of the hard component 230 is brought into contact with (or adheres to) the entire peripheral portions (at least a part of the entire peripheral portions in the axial direction) 31a1 and 32a1 of the outer peripheral surfaces of the plurality of catheter members 30 (the first member 31 and the second member 32 in the present embodiment) arranged at the same position in a predetermined manner so as to apply a predetermined pressure radially inward. With such a configuration, in the present embodiment, due to the shape of the inner peripheral surface 221a of the first hollow portion 221 in the constant cross-sectional area portion 231c of the hard component 230, the fusion surface of the catheter member 30 can be smoothly and stably smoothed without unevenness via the elastic body 220.

The inlet portion 233 of the hard component 230 included in the manufacturing device 200 of the present embodiment has a cross-sectional area larger than that of the outlet portion 234. Therefore, the elastic body 220 and the members 31 and 32 are smoothly inserted into the second hollow portion 231 of the hard component 230 via the inlet portion 233.

The inlet portion 233 of the hard component 230 includes a cross-sectional area decreasing portion 231b whose cross-sectional area gradually decreases along the moving direction. Therefore, by pushing the elastic body 220 and the members 31 and 32 into the second hollow portion 231 of the hard component 230 via the inlet portion 233, the elastic body 220 and the members 31 and 32 can be smoothly moved into the second hollow portion 231. In addition, by inserting the elastic body 220 and the members 31 and 32 into the second hollow portion 231 of the hard component 230 via the inlet portion 233, the elastic body 220 can be brought into an external force application state near the terminal end of the inlet portion 233.

A surface layer 235 for reducing frictional resistance is provided on the inner peripheral surface 231a of the second hollow portion 231 of the hard component 230. Therefore, it is possible to smoothly insert the elastic body 220 and the catheter member 30 into the second hollow portion 231 of the hard component 230 and move the catheter member 30 inside the second hollow portion 231.

The elastic body 220 moves coaxially inside the second hollow portion 231 of the hard component 230. This movement brings at least a part of the entire peripheral portion 231a1 of the inner peripheral surface 231a of the second hollow portion 231 of the hard component 230 into contact with (or adheres to) at least a part of the entire peripheral portion 220a1 of the outer peripheral surface 220a of the elastic body 220. The movement of the elastic body 220 described above is converted into a radial external force with respect to the catheter member 30 via the elastic body 220. The state in which the external force is converted from the coaxial direction to the radial direction in this manner is realized inside the second hollow portion 231 of the hard component 230.

As illustrated in FIGS. 5F and 6, the laser irradiation unit 240 is operated to irradiate the adjacent portion P of the catheter member 30 with the laser beam L (S3 in FIG. 4). Thus, a fusion bonded portion is formed from the outer periphery to the inside of the adjacent portion P between the first member 31 and the second member 32 by heat generation of the first member 31 and the second member 32 and/or heat transfer from the insertion member 210 to the first member 31 and the second member 32. As the timing of the irradiation of the laser beam L, it has been described that the laser beam L is emitted after reaching the above-described external force application state. However, by adjusting the moving speed in the axial direction of the holding mechanism 250 and the output of the laser beam L, the emission of the laser beam L and the external force application state can be substantially simultaneously performed.

When the adjacent portion P of the catheter member 30 is irradiated with the laser beam L, the adjacent portion P can be disposed near the inlet portion 233 of the hard component 230. As described above, a predetermined position of the second hollow portion 231 having a diameter smaller than the outer diameter of the elastic body 220 in a state where no external force is applied is disposed near the inlet portion 233 of the hard component 230 (near the terminal end of the inlet portion 233). Therefore, the elastic body 220 disposed near the inlet portion 233 of the hard component 230 receives the restraining force from the inner peripheral surface 231a of the second hollow portion 231, and the external force application state is appropriately maintained. By irradiating the vicinity of the inlet portion 233 of the hard component 230 with the laser beam L, the adjacent portion P can be appropriately and accurately fused.

In a case where the adjacent portion P is fused along the circumferential direction, the adjacent portion P is irradiated with the laser beam L while the holding mechanism 250 is operated to rotate the elastic body 220, the catheter member 30, and the hard component 230. In a case where the fusion bonded portion is formed over a predetermined range in the axial direction of the catheter member 30, the laser beam L is emitted to the catheter member 30 while the holding mechanism 250 is operated to move the elastic body 220 and the catheter member 30 in the axial direction. In a case where the adjacent portion P is fused along the circumferential direction and the fusion bonded portion is formed over a predetermined range in the axial direction of the catheter member 30, the holding mechanism 250 is operated to rotate and move the elastic body 220, the catheter member 30, and the hard component 230, and the adjacent portion P and the peripheral potion of the adjacent portion P of the catheter member 30 are irradiated with the laser beam L.

The operation of the holding mechanism 250 and the irradiation timing of the laser beam L may be manual, but the operation and the timing can be controlled by a procedure and output stored in advance by the control unit.

In the method for manufacturing a catheter according to the present embodiment, when the elastic body 220 and the catheter member 30 are moved along the axial direction inside the second hollow portion 231 of the hard component 230 and a predetermined portion of the catheter member 30 is disposed at a predetermined position near the inlet portion 233 of the second hollow portion 231 of the hard component 230, the elastic body 220 receives an external force from the inner peripheral surface 231a of the second hollow portion 231 of the hard component 230 and enters an external force application state. Therefore, for example, in a case where the fusion target portion is set over a predetermined range in the axial direction of the catheter member 30, or in a case where the fusion target portion is set at a plurality of different positions in the axial direction of the catheter member 30, by moving the elastic body 220 and the catheter member 30 in the axial direction or stopping the movement as necessary, the irradiation of the predetermined position of the catheter member 30 with the laser beam L can be intermittently or continuously performed, and the catheter member 30 having a smoothly leveled fusion surface can be easily manufactured. Therefore, it is not necessary to set the catheter member 30 in a positioning jig, a dedicated mold, or the like of the manufacturing device every time the fusion bonded portion is formed in the catheter member 30, and thus, it is possible to greatly improve the work efficiency of the manufacturing operation of the catheter 100.

After the fusion of the adjacent portion P is completed, the catheter member 30 is removed from the elastic body 220 and the hard component 230 (S4 in FIG. 4). In the present embodiment, the external force application state by the hard component 230 can be released by moving the elastic body 220 and the catheter member 30 in the axial direction toward the outlet portion 234 side of the hard component 230 and moving the elastic body 220 and the catheter member 30 to the outside of the hard component 230. Thereafter, the catheter member 30 is removed from the elastic body 220.

After the catheter member 30 is detached from the elastic body 220 and the hard component 230, work of attaching the anti-kink protector 21 and the hub 20 is performed, whereby the manufacturing of the catheter 100 illustrated in FIG. 1 can be completed.

As described above, the method for manufacturing a catheter according to the present embodiment can include, in an external force application state in which, in a state where a plurality of catheter members 30 arranged in a predetermined manner via an insertion member 210 are disposed inside a first hollow portion 221 of an elastically deformable elastic body 220 having a laser transmission property and having the first hollow portion 221 through which a plurality of catheter members 30 can be inserted at no load, the elastic body 220 and the plurality of catheter members 30 arranged in a predetermined manner is moved inside a second hollow portion 231 of a hard component 230 having a laser transmission property and harder than the elastic body 220 to bring at least a part of an entire peripheral portion 231a1 of an inner peripheral surface 231a of the second hollow portion 231 of the hard component 230 into contact with (or adheres to) at least a part of an entire peripheral portion 220a1 of an outer peripheral surface 220a of the elastic body 220 so as to apply an external force to the elastic body 220, irradiating the plurality of catheter members 30 arranged in a predetermined manner via the hard component 230 and the elastic body 220 with a laser beam L and fusing the plurality of catheter members 30 arranged in a predetermined manner.

According to the above-described method for manufacturing a catheter, when the laser beam L is emitted, the laser beam L is emitted via the elastic body 220 and the hard component 230 in a state where at least a part of the entire peripheral portion 231a1 of the inner peripheral surface 231a of the second hollow portion 231 of the hard component 230 is brought into contact with (or adheres to) at least a part of the entire peripheral portion 220a1 of the outer peripheral surface 220a of the elastic body 220 to apply an external force to the elastic body 220, whereby a fusion bonded portion can be formed in the catheter member 30. Therefore, in the case of fusion using a heat shrinkable tube, it is necessary to remove the heat shrinkable tube from the catheter member 30 after fusion, but such removal work becomes unnecessary by using the method according to the present embodiment. In addition, the catheter member 30 in which the fusion bonded portion is formed can be easily taken out from the second hollow portion 231 of the hard component 230 when the external force application state is released. Therefore, the elastic body 220 can be used a plurality of times by being reused unlike a heat shrinkable tube used each time. Therefore, in the above-described method for manufacturing a catheter, the risk of damaging the catheter member 30 is extremely small as compared with a conventional method for manufacturing a catheter using a heat shrinkable tube, and labor saving and cost reduction can be achieved while improving quality at the time of manufacturing the catheter 100.

In addition, according to the above-described method for manufacturing a catheter, in a state where the catheter members 30 arranged in a predetermined manner are disposed inside the first hollow portion 221 of the elastic body 220, the elastic body 220 and the catheter members 30 arranged in a predetermined manner are moved inside the second hollow portion 231 of the hard component 230, so that it is possible to bring about an external force application state which is a preparation stage before forming a fusion bonded portion by irradiation with the laser beam L. Furthermore, according to the above-described method for manufacturing a catheter, after the fusion bonded portion is formed, the external force application state can be released by moving the elastic body 220 and the catheter members 30 arranged in a predetermined manner to the outside of the second hollow portion 231 of the hard component 230. Therefore, in the above-described method for manufacturing a catheter, it is possible to uniformly pressurize the catheter member 30 when forming the fusion bonded portion, and it is possible to smoothly level the fusion surface when the catheter member 30 is melted. In addition, it is not necessary to add a complicated manufacturing process for bringing the external force application state to the existing manufacturing method or to add a complicated mechanism to the manufacturing device. Therefore, it is possible to achieve further labor saving and cost reduction when manufacturing the catheter 100.

A method for manufacturing a catheter according to the present embodiment includes, in an external force conversion state in which, in a state where a plurality of catheter members 30 arranged in a predetermined manner on an insertion member 210 is disposed inside a first hollow portion 221 of an elastically deformable elastic body 220 having a laser transmission property and having the first hollow portion 221 through which the plurality of catheter members 30 can be inserted at no load, and the elastic body 220 and the plurality of catheter members 30 arranged in a predetermined manner are disposed inside a second hollow portion 231 of a hard component 230 which has a laser transmission property, has the second hollow portion 231 located coaxially with the insertion member 210, and is harder than the elastic body 220, movement of the elastic body 220 in a coaxial direction inside the second hollow portion 231 of the hard component 230 is converted into a radial external force which brings at least a part of an entire peripheral portion 231a1 of an inner peripheral surface 231a of the second hollow portion 231 of the hard component 230 into contact with (or adheres to) at least a part of an entire peripheral portion 220a1 of an outer peripheral surface 220a of the elastic body 220 and is applied to the plurality of catheter members 30 arranged in a predetermined manner, irradiating adjacent portions P of the plurality of catheter members 30 arranged in a predetermined manner through the hard component 230 and the elastic body 220 with a laser beam L, and fusing the adjacent portions P.

According to the above-described method for manufacturing a catheter, in the external force conversion state in which at least a part of the entire peripheral portion 231a1 of the inner peripheral surface 231a of the second hollow portion 231 of the hard component 230 is brought into contact with (or adheres to) at least a part of the entire peripheral portion 220a1 of the outer peripheral surface 220a of the elastic body 220 so that the movement of the elastic body 220 in the coaxial direction inside the second hollow portion 231 of the hard component 230 is converted into the radial external force applied to the plurality of catheter members 30 arranged in a predetermined manner, the laser beam L can be emitted via the hard component 230 and the elastic body 220 to form the fusion bonded portion in the catheter member 30. Accordingly, in the above-described method for manufacturing a catheter, the risk of damaging the catheter member 30 is extremely small as compared with a conventional method for manufacturing a catheter using a heat shrinkable tube, and labor saving and cost reduction can be achieved while improving quality at the time of manufacturing the catheter 100.

In addition, according to the above-described method for manufacturing a catheter, in a state where the catheter member 30 is disposed inside the first hollow portion 221 of the elastic body 220 and the elastic body 220 and the catheter member 30 are disposed inside the second hollow portion 231 of the hard component 230, a predetermined external force along the coaxial direction is applied to the elastic body 220, so that an external force conversion state, which is a preparation stage before formation of a fusion bonded portion by irradiation with the laser beam L, can be obtained. Furthermore, according to the above-described method for manufacturing a catheter, after the fusion bonded portion is formed, the external force conversion state can be released by releasing the application of the external force along the coaxial direction to the elastic body 220. Therefore, in the above-described method for manufacturing a catheter, it is not necessary to add a complicated manufacturing process for bringing the catheter into an external force conversion state or to add a complicated mechanism to the manufacturing device, as compared with the existing manufacturing method. Therefore, it is possible to achieve further labor saving and cost reduction when manufacturing the catheter 100.

### Configuration Example of Catheter Member

In the method for manufacturing a catheter according to the above-described embodiment, the two parts of the first member 31 and the second member 32 to be the catheter members 30 are arranged in the axial direction, and the adjacent portions P of the two parts are fused by the laser beam L. However, in the method for manufacturing a catheter of the embodiment, the catheter member 30 can be configured as each configuration example described below.

In the first to third configuration examples, the catheter member 30 has a configuration in which a part of the catheter 100 or the catheter 100 and other members are disposed in a predetermined manner. Note that, in the view for explaining each configuration example, the hard component 230 included in the manufacturing device 200 is simply illustrated using an alternate long and short dash line.

### First Configuration Example

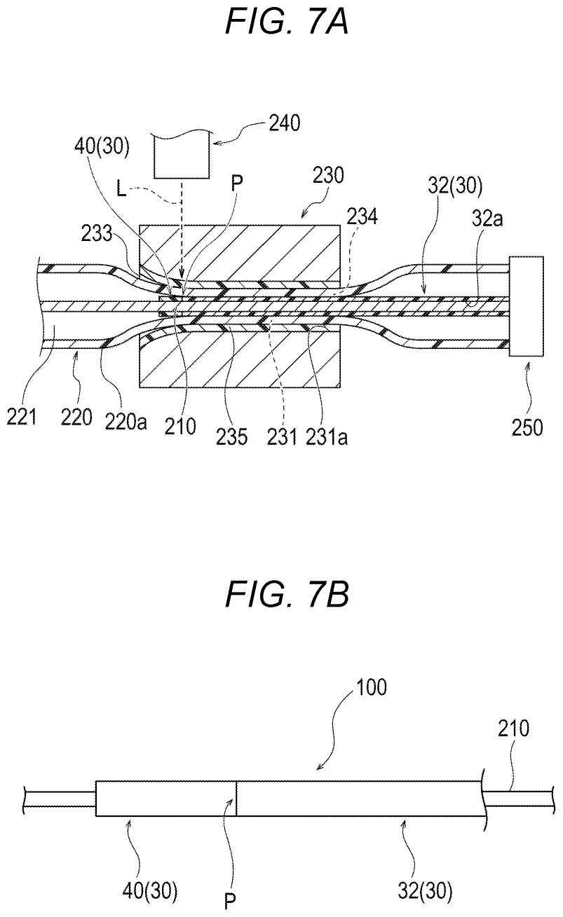

As illustrated in FIGS. 7A and 7B, the catheter member 30 of the first configuration example includes a distal end tip 40 (corresponding to a “first member”) connected to a distal end of the catheter body 10, and a second member 32 constituting the catheter body 10.

When the catheter member 30 of the first configuration example is fused, as illustrated in FIG. 7A, the insertion member 210 is inserted through the distal end tip 40 and the second member 32. The distal end tip 40 and the second member 32 through which the insertion member 210 is inserted are disposed on the hard component 230 of the manufacturing device 200 described above. Then, an external force is applied to the adjacent portion P between the distal end tip 40 and the second member 32 to bring the adjacent portion P into an external force application state, and then the laser beam L is emitted toward the adjacent portion P to form a fusion bonded portion.

In the method for manufacturing a catheter using the catheter member 30 of the first configuration example, as illustrated in FIG. 7B, the catheter 100 in which the distal end tip 40 and the second member 32 forming the catheter body 10 are fused can be manufactured. Note that the cross-sectional shape and the like of the distal end tip 40 are not particularly limited. The distal end tip 40 may have, for example, a non-circular cross-sectional shape.

The method for manufacturing the catheter of the first configuration example can be applied to the catheter as illustrated in FIG. 1, and can also be applied to various types of catheter manufacturing such as a case where a distal end tip is joined to a distal end of a guide wire lumen shaft of a balloon catheter.

### Second Configuration Example

As illustrated in FIGS. 8A and 8B, the catheter member 30 of the second configuration example includes an inner layer and an outer layer constituting the catheter body 10, and a reinforcing body 50 disposed between the inner layer and the outer layer.

In a case where the catheter member 30 of the second configuration example is used, as illustrated in FIG. 8A, the insertion member 210 is inserted into the lumen of a second tube 12 in a state where a first tube 11 (corresponding to the “first member”) serving as an outer layer, the reinforcing body 50, and the second tube 12 (corresponding to the “second member”) serving as an inner layer are arranged in this order from the outside in the radial direction. Together with the second tube 12 through which the insertion member 210 is inserted, the first tube 11 and the reinforcing body 50 are disposed on the hard component 230 of the manufacturing device 200 described above. Then, an external force is applied to the catheter member 30 to bring the catheter member into an external force application state, and then the outer layer (first tube 11), the inner layer (second tube 12), and the laminated portion (adjacent portion P) of the reinforcing body 50 are irradiated with the laser beam L to form a fusion bonded portion. Together with the pressing by the elastic body 220, the fluidity of the first tube 11 is increased by the heat generated by the laser beam L emitted immediately after the elastic body 220 and the first tube 11 is brought into contact with (or adheres to) each other being absorbed by the first tube 11 and the reinforcing body 50, and the resin of the first tube 11 enters the gap of the reinforcing body 50.

As illustrated in FIG. 8B, the method for manufacturing a catheter using the catheter member 30 of the second configuration example can manufacture the catheter 100 in which the reinforcing body 50 is embedded between the inner layer and the outer layer constituting the catheter body 10. Note that the reinforcing body 50 can include, for example, a metal blade, a coil, or the like.

### Third Configuration Example

In the catheter member 30 of the third configuration example, like an aspiration catheter, an IVUS catheter, and an atherectomy catheter, a first member 31 constituting a catheter body 10 and a guide wire shaft 60 (corresponding to a “second member”) including a guide wire lumen 61a are disposed side by side in the radial direction.

FIG. 9A illustrates a state in which the catheter member 30 is disposed on the hard component 230 of the manufacturing device 200. FIG. 9B is an axis-orthogonal cross-sectional view before the first member 31 and the guide wire shaft 60 are fused, and FIG. 9C is an axis-orthogonal cross-sectional view after the first member 31 and the guide wire shaft 60 are fused.

As illustrated in FIGS. 9A and 9B, the catheter member 30 of the third configuration example is disposed on the hard component 230 of the manufacturing device 200 described above in a state where both the members 31 and 60 are arranged in the radial direction by disposing the guide wire shaft 60 in which the insertion member 210 is inserted into the guide wire lumen 61a on the first member 31 in which the insertion member 210 is inserted into the lumen 31a. Then, an external force is applied to the catheter member 30 to bring the external force application state, the adjacent portion P between the first member 31 and the guide wire shaft 60 is irradiated with the laser beam L to form a fusion bonded portion.

In the manufacturing method according to the third configuration example, as illustrated in FIGS. 9B and 9C, a part of the members 31 and 60 located in the adjacent portion P is melted, and the melted portions are fused to each other. During melting and curing, the members 31 and 60 receive a restraining force from the inner peripheral surface 231a of the hard component 230 disposed radially outside the members 31 and 60. Therefore, the members 31 and 60 are melted and cured in a state of receiving a compressive force in the radial direction. Therefore, in the members 31 and 60, the dimension L2 in the long axis direction of the cross section orthogonal to the axis after fusion illustrated in FIG. 9C is smaller than the dimension L2 in the long axis direction of the cross section orthogonal to the axis before fusion illustrated in FIG. 9B.

In the method for manufacturing a catheter using the catheter member 30 of the third configuration example, as illustrated in FIG. 9C, the catheter 100 in which the first member 31 and the guide wire shaft 60 are fused can be manufactured. Note that the catheter member 30 of the third configuration example can also be used, for example, when an intermediate shaft of a balloon catheter and an inner tube shaft including a guide wire lumen are joined.

The catheter in the method for manufacturing a catheter of the present disclosure can include, in addition to a so-called finished product that functions as a catheter after the present manufacturing, an intermediate product that becomes a finished product through a plurality of steps after the present manufacturing, and a product that becomes a part of a finished product or an intermediate product after the present manufacturing.

The detailed description above describes embodiments of a method for manufacturing a catheter. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A method for manufacturing a catheter comprising:
in an external force application state in which, in a state where a plurality of catheter members arranged in a predetermined manner via an insertion member are disposed inside a first hollow portion of an elastically deformable elastic body having a laser transmission property and having the first hollow portion through which a plurality of catheter members can be inserted at no load, the elastic body and the plurality of catheter members arranged in the predetermined manner is moved inside a second hollow portion of a hard component having a laser transmission property and harder than the elastic body to bring at least a part of an entire peripheral portion of an inner peripheral surface of the second hollow portion of the hard component into contact with at least a part of an entire peripheral portion of an outer peripheral surface of the elastic body so as to apply an external force to the elastic body,
irradiating the plurality of catheter members arranged in the predetermined manner via the hard component and the elastic body with a laser beam and fusing the plurality of catheter members arranged in the predetermined manner.

2. The method for manufacturing a catheter according to claim 1, wherein
the second hollow portion of the hard component includes an inlet portion and an outlet portion positioned in a moving direction of the elastic body and the plurality of catheter members arranged in the predetermined manner, and
the inlet portion has a larger cross-sectional area than the outlet portion.

3. The method for manufacturing a catheter according to claim 2, wherein a cross-sectional area of the inlet portion gradually decreases along the moving direction.

4. The method for manufacturing a catheter according to claim 2, further comprising:
emitting the laser beam toward the inlet portion of the hard component.

5. The method for manufacturing a catheter according to claim 1, further comprising:
providing a surface layer for reducing frictional resistance on the outer peripheral surface of the first hollow portion of the elastic body and/or the inner peripheral surface of the hard component.

6. The method for manufacturing a catheter according to claim 1, wherein the plurality of catheter members arranged in the predetermined manner include a first member and a second member arranged in a radial direction.

7. The method for manufacturing a catheter according to claim 1, wherein the plurality of catheter members arranged in the predetermined manner include a first member and a second member arranged in an axial direction.

8. A method for manufacturing a catheter comprising:
in an external force conversion state in which, in a state where a plurality of catheter members arranged in a predetermined manner on an insertion member is disposed inside a first hollow portion of an elastically deformable elastic body having a laser transmission property and having the first hollow portion through which the plurality of catheter members can be inserted at no load, and the elastic body and the plurality of catheter members arranged in the predetermined manner are disposed inside a second hollow portion of a hard component which has a laser transmission property, has the second hollow portion located coaxially with the insertion member, and is harder than the elastic body, movement of the elastic body in a coaxial direction inside the second hollow portion of the hard component is converted into a radial external force which brings at least a part of an entire peripheral portion of an inner peripheral surface of the second hollow portion of the hard component into contact with at least a part of an entire peripheral portion of an outer peripheral surface of the elastic body and is applied to the plurality of catheter members arranged in the predetermined manner,
irradiating predetermined portions of the plurality of catheter members arranged in the predetermined manner through the hard component and the elastic body with a laser beam, and fusing the predetermined portions.

9. The method for manufacturing a catheter according to claim 8, wherein
the second hollow portion of the hard component includes an inlet portion and an outlet portion positioned in a moving direction of the elastic body and the plurality of catheter members arranged in the predetermined manner, and
the inlet portion has a larger cross-sectional area than the outlet portion.

10. The method for manufacturing a catheter according to claim 9, wherein a cross-sectional area of the inlet portion gradually decreases along the moving direction.

11. The method for manufacturing a catheter according to claim 9, further comprising:
emitting the laser beam toward the inlet portion of the hard component.

12. The method for manufacturing a catheter according to claim 8, further comprising:
providing a surface layer for reducing frictional resistance on the outer peripheral surface of the first hollow portion of the elastic body and/or the inner peripheral surface of the hard component.

13. The method for manufacturing a catheter according to claim 8, wherein the plurality of catheter members arranged in the predetermined manner include a first member and a second member.

14. A method for manufacturing a catheter comprising:
disposing via an insertion member, a plurality of catheter members arranged in a predetermined manner inside a first hollow portion of an elastically deformable elastic body having a laser transmission property;
moving the elastic body and the plurality of catheter members arranged in the predetermined manner inside a second hollow portion of a hard component having a laser transmission property and hardness greater than the elastic body, and bringing at least a part of a peripheral portion of an inner peripheral surface of the second hollow portion of the hard component into contact with at least a part of a peripheral portion of an outer peripheral surface of the elastic body and to apply an external force to the elastic body; and
irradiating the plurality of catheter members arranged in the predetermined manner via the hard component and the elastic body with a laser beam and fusing the plurality of catheter members arranged in the predetermined manner.

15. The method for manufacturing a catheter according to claim 14, wherein
the second hollow portion of the hard component includes an inlet portion and an outlet portion, and
the inlet portion has a larger cross-sectional area than the outlet portion.

16. The method for manufacturing a catheter according to claim 15, wherein a cross-sectional area of the inlet portion gradually decreases along the moving direction.

17. The method for manufacturing a catheter according to claim 15, further comprising:
emitting the laser beam toward a vicinity of the inlet portion of the hard component.

18. The method for manufacturing a catheter according to claim 14, further comprising:
providing a surface layer for reducing frictional resistance on the outer peripheral surface of the first hollow portion of the elastic body and/or the inner peripheral surface of the hard component.

19. The method for manufacturing a catheter according to claim 14, wherein the plurality of catheter members arranged in a predetermined manner include a first member and a second member arranged in a radial direction.

20. The method for manufacturing a catheter according to claim 14, wherein the plurality of catheter members arranged in a predetermined manner include a first member and a second member arranged in an axial direction.
